(19) United States
(12) Patent Application Publication
(10) Pub. No.: US 2021/0345926 A1
(43) Pub. Date: Nov. 11, 2021

(54) SMART CUP AND PSYCHOLOGICAL HEALTH MONITORING SYSTEM USING SAME

(71) Applicant: Seung-hee SON, Seoul (KR)

(72) Inventor: Seung-hee SON, Seoul (KR)

(21) Appl. No.: 17/282,968

(22) PCT Filed: Oct. 12, 2018
(86) PCT No.: PCT/KR2018/012003

§ 371 Date: Apr. 5, 2021

(51) Int. Cl. A61B 5/16; G08B 5/36; G08B 3/10; G16Y 20/40; A61B 5/00; A61B 5/0205; A61B 5/024; A61B 5/22; A61B 5/026; A61B 5/389

(52) U.S. Cl. CPC A61B 5/165; A61B 2562/029; G08B 3/10; G16Y 20/40; A61B 5/4845; A61B 5/02055; A61B 5/02438; A61B 5/225; A61B 5/026; A61B 5/389; A61B 5/6887; A61B 5/0008; A61B 5/0022; A61B 5/742; A61B 5/7405; A61B 2562/0271; G08B 5/36

(57) ABSTRACT

A psychological health monitoring system according to an embodiment of the present disclosures includes a smart cup and a terminal. In the psychological health monitoring system, a user's biometric information is collected through a plurality of biosensors, and the user's current state is analyzed and determined on the basis of the collected biometric information, so as to display the user's psychological health/intoxication state through light or sound, so the user can check a psychological health/intoxication state depending on a beverage the user drinks and then correct drinking habits according to the psychological health/intoxication state to maintain good health.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2018/012003 filed on Oct. 12, 2018, the entire contents of which are incorporated herein by reference.

### BACKGROUND

### 1. Technical Field

This disclosure relates to a smart cup and a psychological health monitoring system using the same, and more particularly, to a smart cup capable of collecting biometric information of a user through a plurality of biosensors (sensing modules), analyzing and determining a current state of the user on the basis of the collected biometric information, and displaying a psychological health/intoxication state of the user through light or sound, and a psychological health monitoring system using the same.

### 2. Background Art

In recent years, as the aging of the population progresses due to the development of medical technology and the improvement of living standards, interest in health management of the elderly population is increasing.

However, since various physical diseases and mental stress such as obesity threaten the health of not only the elderly but also the young, interest in health care is not limited to the elderly population, but is a common concern of the entire population.

Here, activity-centered health management may lead to external body changes such as weight loss and muscle strength improvement, but there is a growing awareness of the problem that the effect is temporary, such as returning to the previous body shape when the exercise is stopped.

Accordingly, as the activity-oriented health management is shifted to improvement of lifestyles such as diet management, adequate sleep and posture correction, public consensus is being formed on that if a proper lifestyle is habituated in the long term, health management becomes natural.

In other words, depending on how to manage drinking habits along with food management, the effectiveness of health management may become very high or very low.

Therefore, for efficient health management, it is necessary to check and properly manage psychological health state according to a beverage the user drinks.

### SUMMARY

This disclosure is designed in consideration of the above problems, and the present disclosure is directed to providing a smart cup, which may collect biometric information of a user through a plurality of biosensors (sensing modules), analyze and determine a current state of the user on the basis of the collected biometric information, and display a psychological health/intoxication state of the user through light or sound, and a psychological health monitoring system using the same.

The object of the present disclosure is not limited thereto, and other objects not mentioned herein will be clearly understood by those skilled in the art from the following description.

In order to accomplish the above object, a smart cup and a psychological health monitoring system using the same according to the present disclosure provides a smart cup, which includes a body having a space formed therein to contain a beverage; a sensing module installed to the body to sense biometric information of a user who drinks the beverage; and a control unit configured to store the sensed biometric information to be accumulated, compare the accumulated biometric information and control state information of the user to be displayed in stages according to a comparison value.

In addition, in order to accomplish the above object, a smart cup and a psychological health monitoring system using the same according to the present disclosure provides a smart cup, which includes a body having a space formed therein to contain a beverage; a sensing module installed to the body to sense biometric information of a user who drinks the beverage; and a control unit configured to determine a current state of the user from the sensed biometric information according to a result analyzed based on a preset set value and control state information of the user to be displayed.

In the present disclosure, a light emitting unit configured to emit a light of a preset color to an outer wall of the body by the control unit according to the state information of the user is further included.

In the present disclosure, a sound output unit configured to output a preset sound by the control unit according to the state information of the user is further included.

In the present disclosure, the sensing module includes at least one selected from a vibration sensor for sensing a blood vessel pulsating state of the user, a piezoelectric sensor for sensing a grip strength of the user, a temperature sensor for sensing a body temperature of the user, an electromyogram sensor for sensing an electromyogram of the user, a heart rate sensor for sensing a hand blood flow rate of the user and a humidity sensor for sensing a hand humidity of the user.

In the present disclosure, a communication unit configured to transmit or receive predetermined information to/from a peripheral device by means of a IoT (Internet of Things) function is further included, and the control unit is configured to transmit the state information of the user to the peripheral device through the communication unit.

In addition, in another embodiment of the present disclosure, there is provided a psychological health monitoring system using a smart cup, which includes the smart cup for sensing biometric information as described above, and a terminal configured to output state information of the user received from the smart cup through a predetermined output device and transmit a set value according to the state information of the user to the smart cup,

In the smart cup and the psychological health monitoring system using the same according to the present disclosure, biometric information of a user is collected through a plurality of biosensors (sensing modules), and a current state of the user is analyzed and determined on the basis of the collected biometric information to display a psychological health/intoxication state of the user through light or sound, so that the user my check a psychological health/intoxication state depending on a beverage the user drinks and then correct drinking habits according to the psychological health/intoxication state to maintain good health.

### BRIEF DESCRIPTION OF THE DRAWINGS

FIG. 1 is a perspective view showing a smart cup according to an embodiment of the present disclosure,

FIG. 2 is a block diagram showing the smart cup according to an embodiment of the present disclosure,

FIG. 3 is a sectional view showing the smart cup according to an embodiment of the present disclosure,

FIG. 4 is a perspective view showing a psychological health monitoring system using the smart cup according to an embodiment of the present disclosure, and

FIG. 5 is a block diagram showing the psychological health monitoring system using the smart cup according to an embodiment of the present disclosure.

### DETAILED DESCRIPTION

Hereinafter, a smart cup and a psychological health monitoring system using the same according to the present disclosure will be described in detail with reference to the accompanying drawings.

In adding reference numerals to components of each drawing, it should be noted that the same elements have the same numerals as possible even if they are depicted on different drawings. In addition, in the present disclosure, if it is determined that a detailed description of a related known configuration or function may obscure the subject matter of the present disclosure, the detailed description thereof will be omitted.

In addition, in describing components of the present disclosure, terms such as first, second, A, B, (a), (b) may be used. These terms are only for distinguishing the component from each other, and the nature, order or sequence of the components is not limited by the terms. When a component is described as being “connected”, “coupled” or “accessed” to another component, the component may be directly connected or accessed to another component, but it should be understood that still another component may be “connected”, “coupled” or “accessed” between these components.

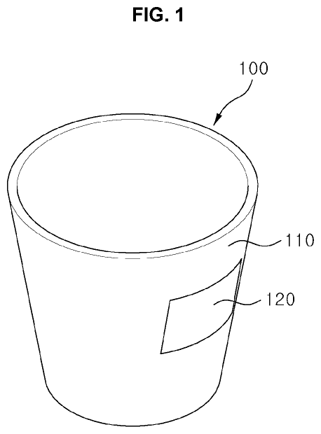

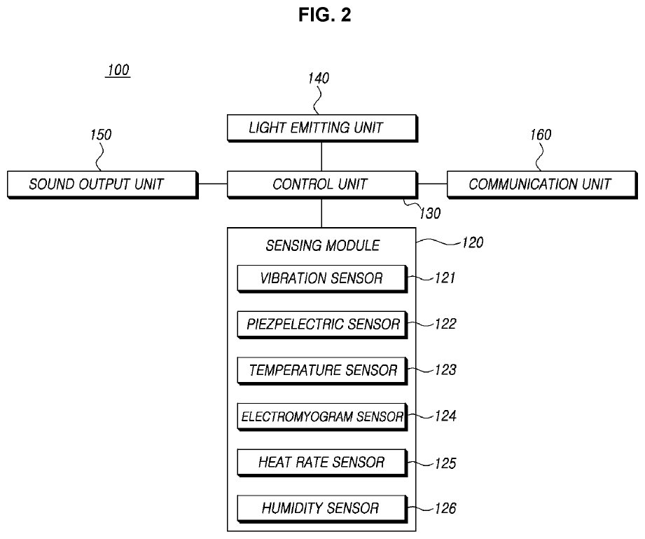

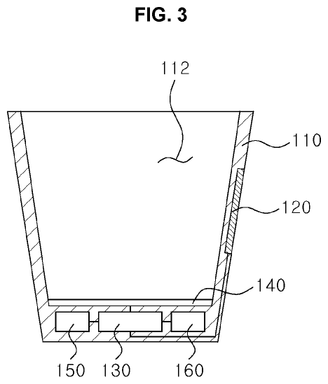

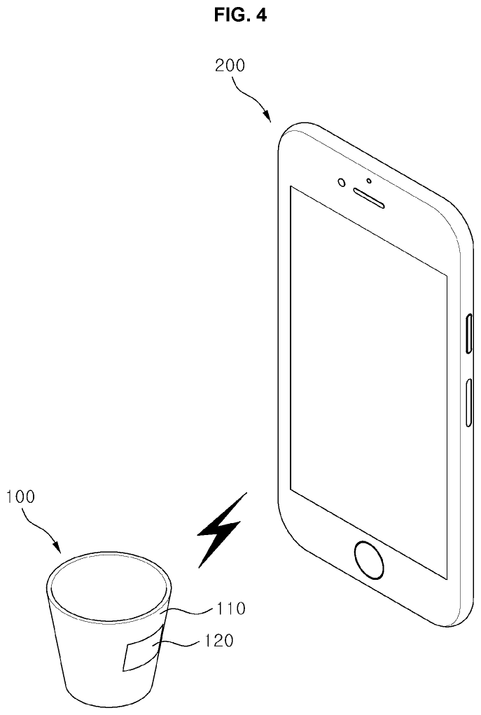

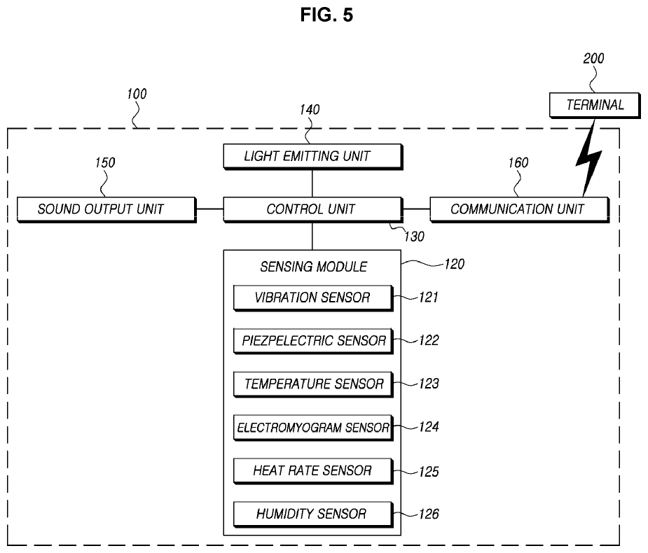

FIG. 1 is a perspective view showing a smart cup according to an embodiment of the present disclosure, FIG. 2 is a block diagram showing the smart cup according to an embodiment of the present disclosure, FIG. 3 is a sectional view showing the smart cup according to an embodiment of the present disclosure, FIG. 4 is a perspective view showing a psychological health monitoring system using the smart cup according to an embodiment of the present disclosure, and FIG. 5 is a block diagram showing the psychological health monitoring system using the smart cup according to an embodiment of the present disclosure.

A smart cup 100 of the present disclosure includes a body 110 having a space 112 formed therein to contain a beverage; a sensing module 120 installed to the body 110 to sense biometric information of a user who drinks the beverage; and a control unit 130 configured to store the sensed biometric information to be accumulated, compare the accumulated biometric information and control state information of the user to be displayed in stages according to a comparison value.

In addition, the smart cup 100 of the present disclosure includes a body 110 having a space 112 formed therein to contain a beverage; a sensing module 120 installed to the body 110 to sense biometric information of a user who drinks the beverage; and a control unit 130 configured to determine a current state of the user from the sensed biometric information according to a result analyzed based on a preset set value and control state information of the user to be displayed.

The body 110 has a cylindrical shape made of transparent material so that its lower side is sealed and its upper side is opened to form the space 112 for containing a beverage such as water, soju, beer, juice, tea, etc., and the sensing module 120 for sensing biometric information is installed to an outer surface of the body 110 where a part of the body of the user comes into contact.

Here, the body 110 may be formed in a polygonal column shape made of a translucent material or an opaque material, and may have a double cup structure with an outer wall and an inner wall.

The sensing module 120 senses biometric information of a user who drinks the beverages, and includes at least one of a vibration sensor 121 for sensing a blood vessel pulsating state of the user, a piezoelectric sensor 122 for sensing a grip strength of the user, a temperature sensor 123 for sensing a body temperature of the user, an electromyogram sensor 124 for sensing an electromyogram of the user, a heart rate sensor 125 for sensing a hand blood flow rate of the user and a humidity sensor 126 for sensing a hand humidity of the user.

In the present disclosure, the sensing module 120 is installed to the outer surface of the body 110 so that it comes into contact with a part of the body of the user. A change amount of a photoplethysmography (PPG), a skin temperature (SKT), a galvanic skin response (GSR), a heart rate (HR), or the like is measured by the sensing module 120, each measured sensor value is transmitted to the control unit 130.

The control unit 130 analyzes and determines a current state of the user from the sensed biometric information, and controls a psychological health/intoxication state information of the user to be displayed through a light emitting unit 140 or a sound output unit 150.

In other words, the control unit 130 classifies and determines a current psychological health state of the user into active, comfortable, drowsy and anxious states according to the analysis result of each sensor value transmitted from the sensing module 120, and controls the psychological health state information to be displayed through the light emitting unit 140 or the sound output unit 150.

At this time, the control unit 130 stores the sensor values to be accumulated in a memory, determines the current psychological health state of the user into active, drowsy, anxious and comfortable states based on the preset set value through a calculation process according to the psychological health state analysis and judgment algorithm, and controls the current state to be displayed accordingly through the light emitting unit 140 or the sound output unit 150.

In addition, the control unit 130 classifies and determines a current intoxication state of the user in preset stages according to the analysis result of each sensor value transmitted from the sensing module 120, and controls the intoxication state information of the user according to the classification and determination to be displayed through the light emitting unit 140 or the sound output unit 150.

In addition, the control unit 130 may store the sensor values to be accumulated in the memory, compare the accumulated sensor values, and control the state information of the user to be displayed in stages through the light emitting unit 140 or the sound output unit 150 according to the comparison value.

The light emitting unit 140 emits a light of a preset color to the outer wall of the body 110 by the control unit 130 according to the psychological health state information of the user or the intoxication state information of the user, and may emit light of various colors such as red, green and yellow in stages according to the psychological health state information of the user or the intoxication state information of the user through the body 110 made of a transparent material.

In other words, the light emitting unit 140 emits any one of various preset colors such as red, green and yellow through the body 110 made of a transparent material according to the psychological health state information of the user, or changes the brightness of the light of the color according to the intoxication state information of the user and emits any one of the lights with different brightness through the body 110 made of a transparent material.

The sound output unit 150 outputs a preset sound by the control unit 130 according to the psychological health state information of the user or the intoxication state information of the user, and outputs various sounds such as ‘active’, ‘drowsy’, ‘anxious’ and ‘comfortable’ according to the psychological health state information of the user or the intoxication state information of the user.

That is, the sound output unit 150 outputs one of various preset sounds such as ‘active’, ‘drowsy’, ‘anxious’ and ‘comfortable’ according to the psychological health state information of the user, or outputs any one of sounds such as ‘first-stage intoxication’, ‘second-stage intoxication’ and ‘third-stage intoxication’ according to the intoxication state information of the user.

The communication unit 160 transmits and receives predetermined information to/from a peripheral device through an IoT (Internet on Things) function, and may transmit the psychological health state information of the user or the intoxication state information of the user to the peripheral device.

In a psychological health monitoring system using the smart cup 100 according to another embodiment of the present disclosure, the smart cup 100 is the same as described above and thus will not be described in detail.

The terminal 200 outputs the psychological health state information of the user or the intoxication state information of the user received from the smart cup 100 through a predetermined output device, and transmits a set value according to the psychological health state information of the user or the intoxication state information of the user to the smart cup 100.

Here, the set value may be a stored value stored in a memory, a sensor value initially transmitted from the sensing module 120, or an input value input from the terminal 200.

That is, the terminal 200 outputs the psychological health state information of the user or the intoxication state information of the user received from the smart cup 100 by means of the communication unit 160 through an output device such as a display unit or a print, and transmits the set value, which serves as a criterion to determine to determine the intoxication state of the user into active, drowsy, anxious or comfortable states, to the smart cup 100.

Therefore, the smart cup 100 determines the result obtained by analyzing each sensor value transmitted from the sensing module 120 as an active, drowsy, anxious or comfortable state according to the preset set value for the psychological health state of the user, and displays the set value accordingly through the light emitting unit 140 or the sound output unit 150.

In addition, the control unit 130 determines the result obtained by analyzing each sensor value transmitted from the sensing module 120 in stages according to the preset set value for the intoxication state of the user, and displays the set value through the light emitting unit 140 or the sound output unit 150 accordingly.

For example, in a state where ordinary biometric information of the user at ordinary day is stored as reference data, the control unit 130 compares the biometric information of the user transmitted from the sensing module 120 with the ordinary biometric information to determine the biometric information of the user into an active, drowsy, anxious or comfortable state according to the preset set value for the psychological health state of the user based on the number of biometric information out of a preset range or the size of biometric information out of a preset range. Alternatively, in a state where ordinary biometric information of the user at ordinary day is stored as reference data, the control unit 130 compares the biometric information of the user transmitted from the sensing module 120 with the ordinary biometric information to determine the intoxication information of the user in stages according to the preset set value for the intoxication state of the user based on the number of biometric information out of a preset range or the size of biometric information out of a preset range.

As another example, the control unit 130 stores sensor values to be accumulated in the memory, compares the accumulated sensor values, and controls the intoxication state information of the user to be displayed according to the comparison value by emitting a light with different brightness in stages through the light emitting unit 140 or outputting any one of the preset sounds through the sound output unit 150.

As described above, since the smart cup 100 and the psychological health monitoring system using the same according to the present disclosure collects the biometric information of the user through a plurality of biosensors (the sensing modules 120) and analyzes and determines a current state of the user from the collected biometric information to display the psychological health/intoxication state of the user through light or sound, it is possible to maintain health by checking the psychological health/intoxication state according to the beverage the user drinks and correcting drinking habits of the user afterwards according to the psychological health/intoxication state.

In the above, just because all components constituting the embodiment of the present disclosure are coupled into one or described as operating in a combined state, the present disclosure is not necessarily limited to this embodiment. That is, within the scope of the present disclosure, at least one of all components may be selectively combined and operate in a combined state.

In addition, the terms such as “comprise”, “include” or “have” described above mean that the corresponding component may be included, unless otherwise stated, and thus it should be construed as that other components are not excluded but can be further included. All terms, including technical or scientific terms, unless otherwise defined, have the same meaning as commonly understood by those skilled in the art. Generally used terms, such as terms defined in the dictionary, should be interpreted as being consistent with the meaning in the context of the related technology, and should not be interpreted in an ideal or excessively formal meaning unless explicitly defined in the present disclosure.

The above explanation is merely an illustrative explanation of the technical idea of the present disclosure, and can be changed or modified in various ways by those skilled in the art without departing from the essential characteristics of the present disclosure. Accordingly, the embodiments disclosed in the present disclosure are not intended to limit the technical idea of the present disclosure, but to describe, and the scope of the technical idea of the present disclosure is not limited by these embodiments. The scope of the present disclosure should be interpreted by the appended claims, and all technical ideas within the equivalent scope should be construed as being included in the scope of the present disclosure.

1. A psychological health monitoring system using a smart cup, comprising:
a smart cup including a body having a space formed therein to contain a beverage, a sensing module installed to the body to sense biometric information of a user who drinks the beverage, a control unit configured to store the sensed biometric information to be accumulated, compare the accumulated biometric information and control state information of the user to be displayed in stages according to a comparison value, and a communication unit configured to transmit or receive predetermined information to/from a peripheral device by means of a IoT (Internet of Things) function; and
a terminal configured to output psychological health state information of the user or intoxication state information of the user received from the smart cup through a predetermined output device and transmit a set value according to the psychological health state information of the user or the intoxication state information of the user to the smart cup,
wherein the sensing module includes at least one selected from a vibration sensor for sensing a blood vessel pulsating state of the user, a piezoelectric sensor for sensing a grip strength of the user, a temperature sensor for sensing a body temperature of the user, an electromyogram sensor for sensing an electromyogram of the user, a heart rate sensor for sensing a hand blood flow rate of the user and a humidity sensor for sensing a hand humidity of the user; and
the control unit is configured to transmit the state information of the user to the peripheral device through the communication unit.

2. The psychological health monitoring system using a smart cup according to claim 1, wherein the smart cup comprises a light emitting unit configured to emit a light of a preset color to an outer wall of the body by the control unit according to the state information of the user.

3. The psychological health monitoring system using a smart cup according to claim 1, wherein the smart cup comprises a sound output unit configured to output a preset sound by the control unit according to the state information of the user.
